(19) United States
(12) Patent Application Publication
(10) Pub. No.: US 2007/0135679 A1
(43) Pub. Date: Jun. 14, 2007

(54) Colonoscopic device stabilizer

(75) Inventors: John V. Hunt, Cincinnati, OH (US); Michael S. Cropper, Edgewood, KY (US); Richard C. Smith, Milford, OH (US); Richard F. Schwemberger, Cincinnati, OH (US); John P. Measamer, Cincinnati, OH (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Ethicon Endo-Surgery, Inc.

(21) Appl. No.: 11/301,912

(22) Filed: Dec. 13, 2005

(51) Int. Cl. A61B 1/00

(52) U.S. Cl. 600/102; 600/114

(57) ABSTRACT

A colonoscopic device stabilizer for stabilizing a colonoscopic device when inserted in an anus of a patient. The stabilizer includes a body adapted for receipt within the anus of a patient. The body has an opening sized and shaped for receiving the colonoscopic device when the device is inserted in the anus of the patient. The stabilizer also includes a clamp attached to the body selectively moveable between a locked position in which the clamp resists movement of the colonoscopic device relative to the body and an unlocked position in which the clamp permits movement of the colonoscopic device relative to the body.

### BACKGROUND OF THE INVENTION

The present invention relates generally to colonoscopic equipment, and more particularly to a stabilizer for stabilizing a colonoscopic device.

Colonoscopic devices are medical devices used for examining and performing surgery inside a bowel of a patient. Colonoscopic devices typically include an elongate member for entering the body through a patient's anus and for extending into the patient's bowel so the interior of the bowel can be visually examined or operated on without making large incisions through the patient's tissue to gain access. Thus, colonoscopic devices permit less invasive examination and surgeries than conventional examination and surgical methods. Many colonoscopic devices may be articulated or bent to follow the bowel to a particular site of interest. One type of colonoscopic device is referred to as a colonoscope, which allows a user to view a patient's bowel to make visual examinations or to view the bowel where surgery is being performed. Other types of colonoscopic devices include suturing equipment, scalpels, and forceps. A colonoscopist guides the colonoscopic device through the patient's bowel to position a functional end or tip of the device at the site of interest so the device can perform the task for which it is intended.

A colonoscopist usually must use one or both hands to guide the colonoscopic device into position. For example, when guiding a colonoscopic device into a patient's bowel through his or her anus, the colonoscopist must use one hand adjacent the patient's anus to guide the colonoscope into the anus and hold the colonoscope in position. Frequently, the patient's rectal muscles tend to move the colonoscope involuntarily. Thus, the colonoscopist must keep one hand on the device to hold it in position against the forces of the patient's involuntary reflexes. Because the colonoscopist's hand is needed to guide the device and hold it in position, the hand is not available for performing other tasks such as operating video recording equipment to record the views seen through the device or to operate other devices used to perform diagnosis therapy or surgery. Similarly, when guiding an endoscopic device, which is a more generalized device than a colonoscopic device for insertion through orifices other than a patient's anus (e.g., natural orifices or through incisions), into a patient, the endoscopist faces similar problems to those described above. Accordingly, there is a need for colonoscopic equipment which will guide colonoscopic devices into a patient and temporarily hold them in position within the patient.

In addition, when a colonoscopist guides colonoscopic devices into a patient's anus, the colonoscopic equipment contacts the soft tissue of the patient's anus, potentially damaging the soft tissue. Thus, there is a need for a colonoscopic equipment for preventing contact between the patient's anus and the colonoscopic device to eliminate a potential for damage to the patient's anus.

### SUMMARY OF THE INVENTION

Briefly, the present invention includes a colonoscopic device stabilizer for stabilizing a colonoscopic device when inserted in an anus of a patient. The stabilizer comprises a body adapted for receipt within the anus of a patient. The body has an opening sized and shaped for receiving the colonoscopic device when the device is inserted in the anus of the patient. In addition, the stabilizer comprises a clamp attached to the body selectively moveable between a locked position in which the clamp resists movement of the colonoscopic device relative to the body and an unlocked position in which the clamp permits movement of the colonoscopic device relative to the body.

In another aspect, the invention includes a method of inserting a colonoscopic device into an anus of a patient. The method comprises positioning a stabilizer into the anus of the patient, inserting the colonoscopic device into the stabilizer, and advancing the colonoscopic device through the stabilizer.

In still another aspect, the invention includes a method of inserting an endoscopic device into an orifice in a patient. The method comprises positioning a stabilizer into the orifice of the patient, inserting the endoscopic device into the stabilizer, and advancing the endoscopic device through the stabilizer.

Other features of the present invention will be in part apparent and in part pointed out hereinafter.

### BRIEF DESCRIPTION OF THE DRAWINGS

FIG. 1 is a perspective of a colonoscopic device stabilizer of a first embodiment of the present invention;

FIG. 2 is a cross section of the colonoscopic device stabilizer of the first embodiment;

FIG. 3 is a cross section of the stabilizer similar to FIG. 2 but with a clamp of the stabilizer in a locking position;

FIG. 4 is a perspective of a colonoscopic device stabilizer of a second embodiment of the present invention; and

FIG. 5 is a perspective of a colonoscopic device stabilizer of a third embodiment of the present invention.

FIG. 6 is a partially transparent perspective of a colonoscopic device stabilizer of a fourth embodiment of the present invention.

FIG. 7 is a horizontal plan in partial section of a colonoscopic device stabilizer of a fifth embodiment of the present invention.

FIG. 8 is a horizontal plan in partial section of a colonoscopic device stabilizer of a sixth embodiment of the present invention.

FIG. 9 is a partially transparent perspective of a colonoscopic device stabilizer of a seventh embodiment of the present invention.

FIG. 10 is a partially transparent perspective of a colonoscopic device stabilizer of a eighth embodiment of the present invention.

FIG. 11 is a partially transparent perspective of a colonoscopic device stabilizer of an ninth embodiment of the present invention.

FIG. 12 is a perspective of a colonoscopic device stabilizer of a tenth embodiment of the present invention.

Corresponding reference characters indicate corresponding parts throughout the several views of the drawings.

### DETAILED DESCRIPTION OF THE PREFERRED EMBODIMENT

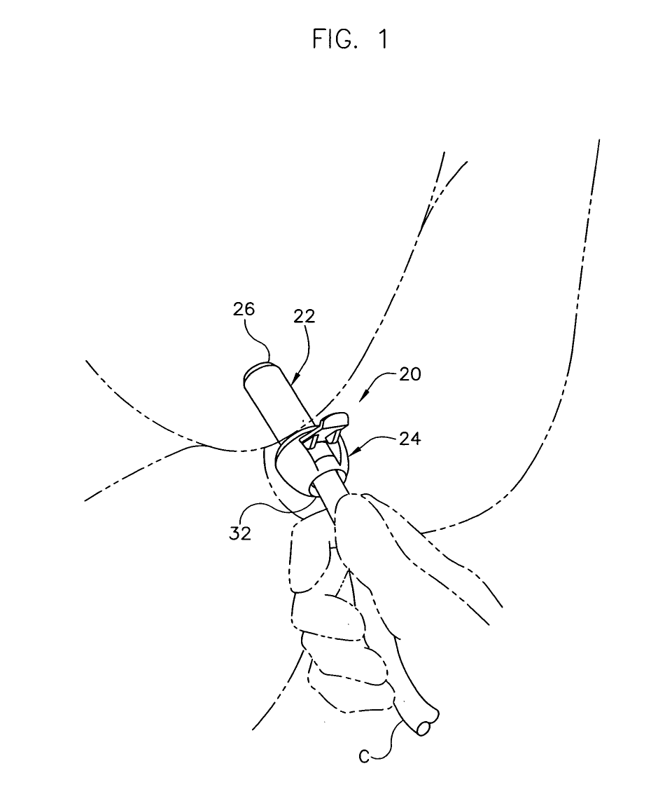

Referring now to the drawings and in particular to FIG. 1, a colonoscopic device stabilizer of the present invention is designated in its entirety by the reference numeral 20. The stabilizer 20 is intended for use with a colonoscopic device such as a colonoscope C. The stabilizer 20 generally comprises a body, generally designated by 22, and a clamp, generally designated by 24.

The body 22 is adapted for receipt within an anus of a patient as shown in FIG. 1. In one embodiment, the body 22 is generally cylindrical. Although the body 22 may have other dimensions without departing from the scope of the present invention, in one embodiment the body has a length of between about four centimeters and about forty centimeters, and a diameter of between about five millimeters and about thirty millimeters. In one embodiment, the body 22 has a tapered or beveled tip 26 for reducing a potential for injuring the patient's anus as the body is inserted. Those of ordinary skill in the art will appreciate that the overall size and shape of the body 22 may be modified to fit a variety of patient anus sizes.

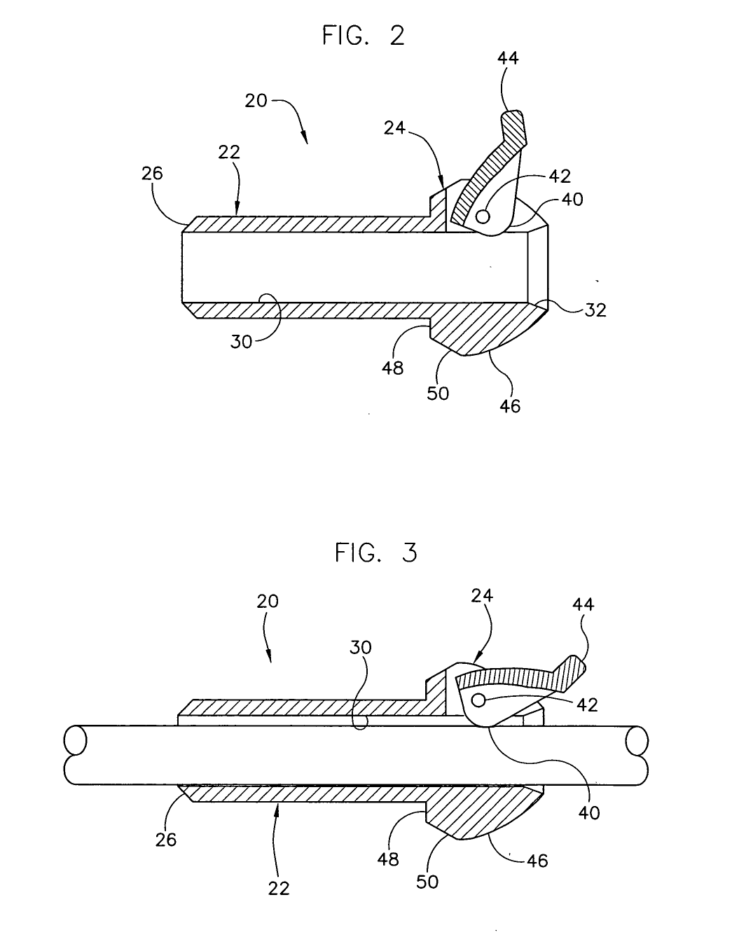

As illustrated in FIG. 2, the body includes an opening 30 sized and shaped for receiving the colonoscopic device C when the device is inserted into the anus of the patient. Although the opening 30 may have other shapes and sizes without departing from the scope of the present invention, in one embodiment the opening is fully enclosed (i.e., surrounded by the body) and circular. Further, the opening 30 of this embodiment has a diameter between about two millimeters and about 25 millimeters. The opening 30 may include a beveled edge 32 at one end to provide a wider entry port for easing insertion of the colonoscopic device C into the opening. Although the body 22 may be made of other materials without departing from the scope of the present invention, in one embodiment the body is made from a polymer such as polycarbonate or Kraton elastomer available from Shell Oil Company of Houston, Tex. It is envisioned that the opening 30 of the stabilizer 20 may also include a seal system (not shown) to reduce insufflation or gas loss. Further, the stabilizer 20 may have more than one opening 30 for accommodating additional endoscopic devices without departing from the scope of the present invention. In addition, it is envisioned the stabilizer 20 of the present invention may be used in combination with a conventional shape locking tube (not shown) to further stabilize the endoscopic device. Alternatively, it is envisioned a shape locking tube may be integrally formed with the stabilizer 20.

As further illustrated in FIG. 2, the clamp 24 in one embodiment comprises a cam 40 pivotally mounted on a pin 42 for selectively movement between an unlocked position as shown in FIG. 2 in which the clamp permits movement of the colonoscopic device C in the opening 30 of the body 22 and a locked position as shown in FIG. 3 in which the clamp resists movement of the colonoscopic device relative to the body. The clamp 24 also includes a lever 44 integrally formed with the cam 40 for applying leverage to the cam to pivot it between the locked position and the unlocked position. As will be appreciated by those skilled in the art, the clamp 24 may include a collar 46 on which the cam 40 is mounted. In one embodiment, this collar 46 is integrally formed with the body 22. A shoulder 48 is provided between the collar 46 and the body 22 to reduce a potential for inserting the stabilizer 20 too far into the patient's anus so that the clamp 24 is difficult to operate. Further, in one embodiment an outer edge of shoulder 48 has a large chamfer 50 to reduce a potential for injuring the patient's anus when the shoulder makes contact.

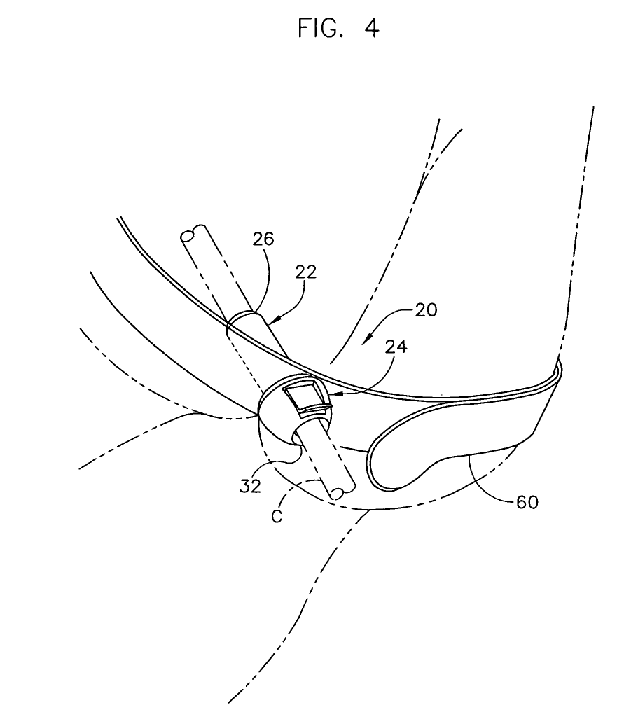

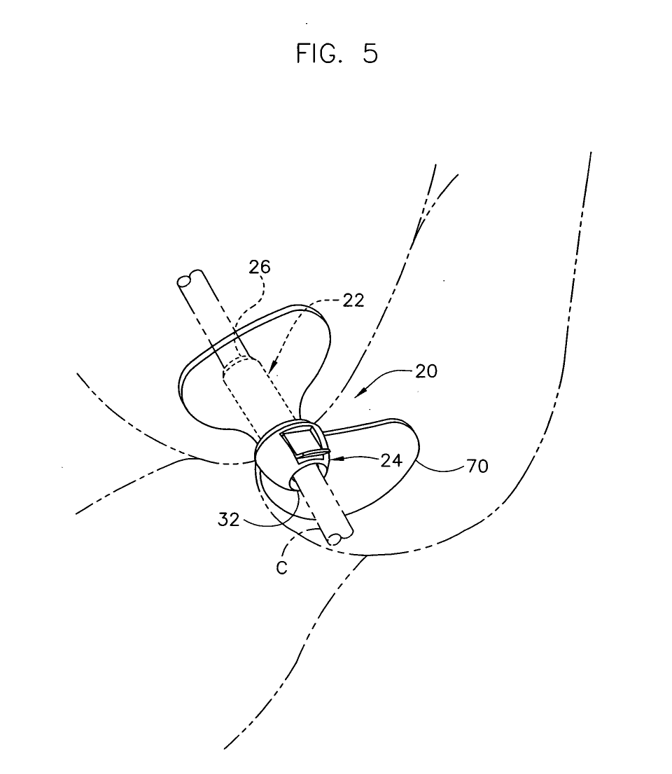

The stabilizer 20 of the present invention may be held in place in several different ways. For example, if the patient is awake and unsedated, the patient may use his or her hand to hold the stabilizer in place as shown in FIG. 1. In other instances, the body 20 may be positioned between the patient's upper and lower teeth and a strap 60 may be used to hold the stabilizer 20 in place as shown in FIG. 4. The strap at least partially encircles the patient's waist and may be fastened, such as with a gripper fastener, hook and loop fasteners (e.g., Velcro fasteners), or with a buckle (not shown). It is also envisioned that the stabilizer 20 may be held in position with an adhesive strip or tape 70 as shown in FIG. 5. Although other types of tape 70 may be used without departing from the scope of the present invention, in one embodiment the tape is made of a flexible plastic or woven fabric coated with a pressure-sensitive, medical grade adhesive suitable for use in adhesive bandages. In one embodiment, the tape adhesive is a hypoallergenic hot melt adhesive. Emulsion adhesives may also be used provided the adhesive application station includes means for drying the adhesive after application. An example of such an adhesive strip is described in U.S. Pat. No. 4,622,089. Although the tape 70 may have other configurations, in one embodiment the tape has the “hourglass” or “butterfly” shape shown in FIG. 5 so the tape seats against each buttock. It is also envisioned that the stabilizer 20 may be held in position with adhesive, straps, sutures, clips or staples. Still further, the stabilizer 20 may be attached to the patient's bed or to a stand without departing from the scope of the present invention.

The stabilizer 20 described above may be used to insert a colonoscopic device (e.g., a colonoscope) into an anus of a patient. The colonoscopist positions the stabilizer 20 in the anus of the patient, inserts the colonoscopic device C into the stabilizer opening 30, and advances the colonoscopic device through the stabilizer. Once the colonoscopic device C is in a desired position with the tip of the colonoscopic device in a desired position within the patient, the clamp 24 may be moved from the unlocked position to the locked position by rotating the lever 44 from the position shown in FIG. 2 to the position shown in FIG. 3. As the lever 44 is rotated, the cam 40 rotates about the pin 42 so it engages the surface of the colonoscopic device to hold the colonoscopic device relative to the stabilizer 20 so it stays in position in the patient. As described above, the stabilizer 20 may be attached to the patient prior to inserting the colonoscopic device into the stabilizer by one of the methods described above.

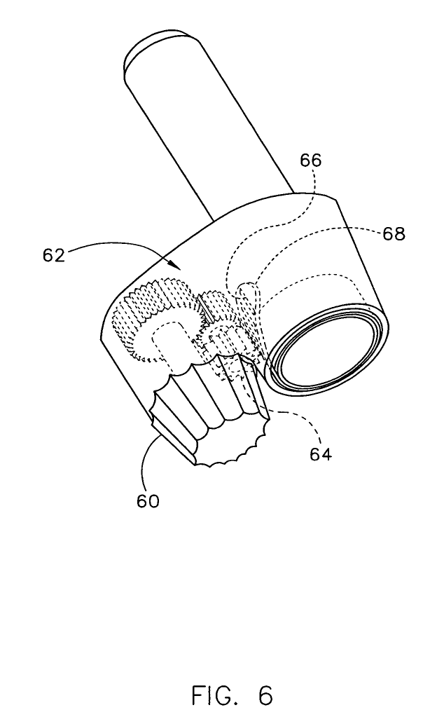

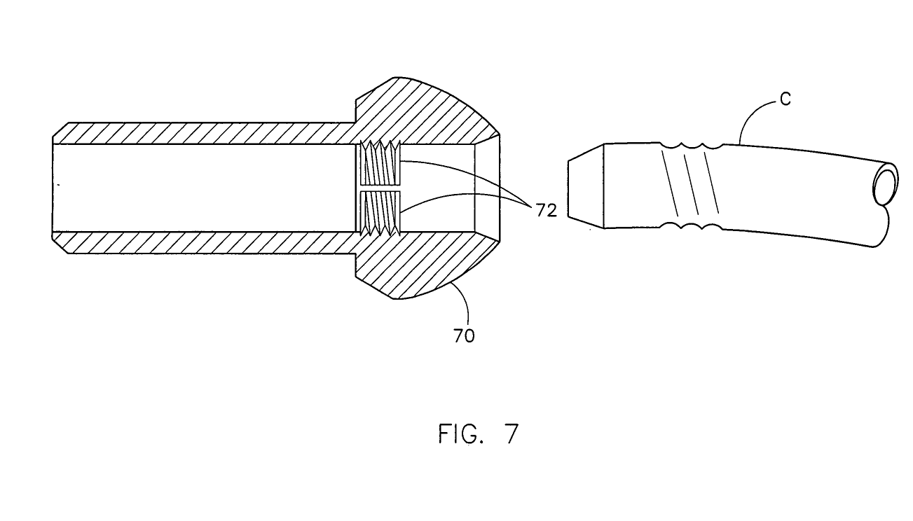

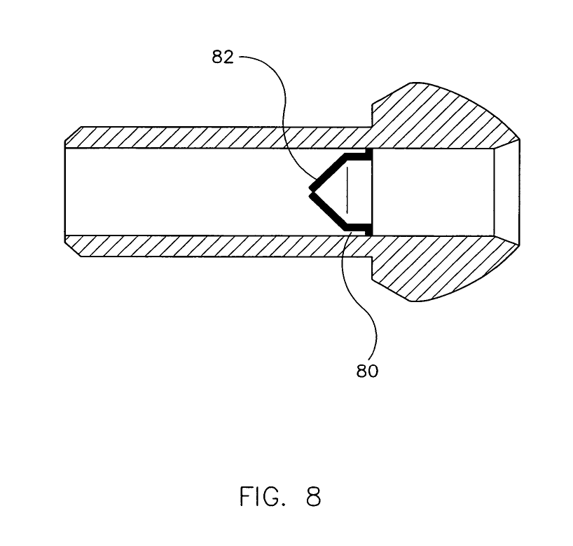

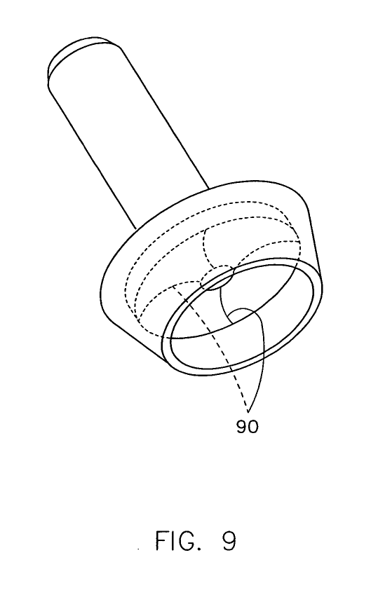

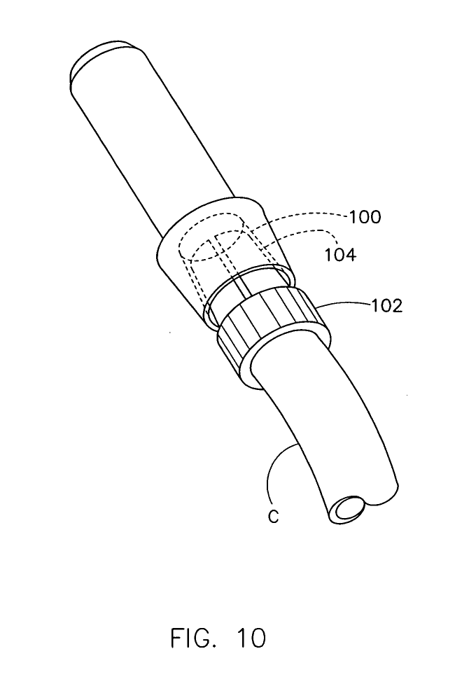

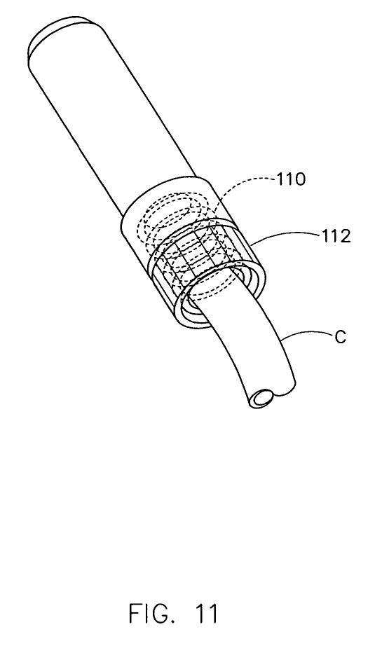

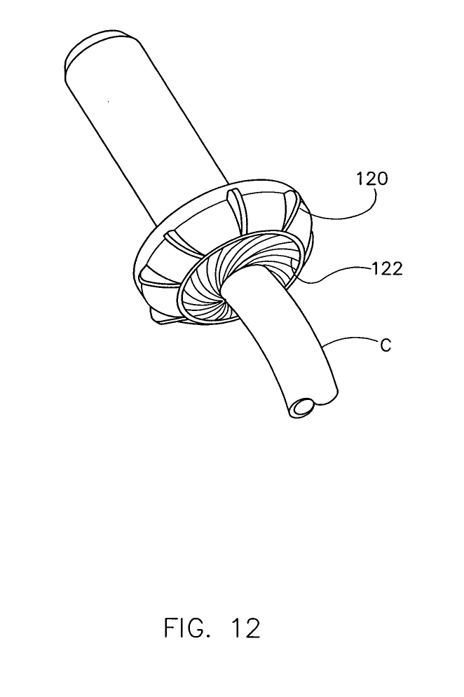

The clamp 24 may have other configurations without departing from the scope of the present invention. For example, the clamp 24 may have a strap clamp configuration as illustrated in FIG. 6, a collet configuration as illustrated in FIG. 7, a trocar-type configuration as illustrated in FIGS. 8 and 9, a rubber stopper configuration as illustrated in FIG. 10, a compressive sleeve lock configuration as illustrated in FIG. 11, an iris-type lock configuration as illustrated in FIG. 12, or a detent configuration without departing from the scope of the present invention. In the strap clamp configuration of FIG. 6, a knob 60 is provided for actuating the clamp. The knob 60 is operatively connected to a train of gears, generally designated 62, that drives a pinion 64. The pinion engages a rack 66 formed by a series of slots in a strap 68. The strap 68 is selectively tightened or loosened around the colonoscopic device C (not shown) by turning the knob 60. In the collet configuration of FIG. 7, an internally threaded nut 70 engages fingers 72 so that the fingers are compressed against the colonoscopic device C (not shown) when the nut is turned in one direction (i.e., clockwise) and released when the nut is turned in an opposite direction. In the trocar-type configuration shown in FIG. 8, a flexible duck bill seal 80 is formed for receiving the colonoscopic device C (not shown). The seal 80 has a slit 82 which opens as the seal deforms to receive the colonoscopic device C. In the configuration shown in FIG. 9, a plurality of flexible leaves 90 are provided around the opening. The leaves 90 deform to receive the colonoscopic device C. In the stopper configuration shown in FIG. 10, the opening 36 includes a tapered portion 100. A stopper 102 having fingers 104 is received within the tapered portion 100. When the stopper 102 is pushed into the tapered portion 100 of the opening 36, the fingers 104 grip the colonoscopic device C. In the compressive sleeve lock configuration illustrated in FIG. 11, an internal spring 110 grasps the colonoscopic device C to hold it in position. The force applied by the spring 110 on the colonoscopic device C may be adjusted by turning a knob 112 attached to one end of the spring. When the knob 112 is turned in one direction, an inner diameter of the spring is reduced thereby increasing the force applied by the spring, and when the knob is turn in an opposite direction, the inner diameter of the spring is increased thereby decreasing the force applied by the spring. In the iris configuration shown in FIG. 12, the user turns a collar 120 surrounding an iris 122 to open and close the iris around the colonoscopic device C. Because each of these clamp configurations is generally conventional, they will not be described in further detail. As will be appreciated by those skilled in the art, various changes and modifications may be made to the configurations described above without departing from the scope of the present invention.

As will be appreciated by those skilled in the art, the stabilizer 20 of the present invention, stabilizes the colonoscopic device C to resist movement during therapy, diagnosis or surgery. With the colonoscopic device C stabilized by the stabilizer 20 of the present invention, the colonoscopist's hands are free to perform other tasks such as approximating tissue, suturing, or cutting tissue. Therefore, those skilled in the art will appreciate that the stabilizer 20 of the present invention has several advantages over prior art devices. As will be appreciated by those skilled in the art, the stabilizer 20 of the present invention may be used to stabilize endoscopic devices other than colonoscopic devices and may be used to stabilize devices in natural orifices such as an anus or vagina, in wounds and in incisions.

In one embodiment, the stabilizer device described above may be used to perform intra-abdominal surgery. The stabilizer device is inserted in an orifice in the patient and secured relative to the patient once the device is in the proper position. The endoscopic device is inserted in the stabilizer device and advanced through the stabilizer device so the endoscopic device extends into an internal cavity in the patient such as an abdominal cavity or a thoracic cavity. An incising instrument is inserted through the endoscopic device, advanced to a desired location within the cavity and an incision is made in an internal wall of the cavity. The endoscopic device may be locked in position relative to the stabilizer device to permit inspection of the cavity and permit a surgical procedure to be performed within the cavity. The endoscopic device may be repositioned or removed by unlocking the stabilizer device. When the procedure is complete, the stabilizer may be unlocked and all the endoscopic devices and instruments may be removed. The orifice mentioned above in this intra-abdominal surgery procedure may be a natural orifice such as an anus of the patient.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions without departing from the scope of the invention, it is intended that all matter contained in the above description or shown in the accompanying drawings shall be interpreted as illustrative and not in a limiting sense.

What is claimed is:

1. A colonoscopic device stabilizer for stabilizing a colonoscopic device when inserted in an anus of a patient, said stabilizer comprising:
a body adapted for receipt within the anus of a patient, said body having an opening sized and shaped for receiving the colonoscopic device when said device is inserted in the anus of the patient; and
a clamp attached to the body selectively moveable between a locked position in which the clamp resists movement of the colonoscopic device relative to the body and an unlocked position in which the clamp permits movement of the colonoscopic device relative to the body.

2. A colonoscopic device stabilizer as set forth in claim 1 wherein said body is generally cylindrical.

3. A colonoscopic device stabilizer as set forth in claim 2 wherein said body has a length between about four centimeters and about forty centimeters.

4. A colonoscopic device stabilizer as set forth in claim 2 wherein said body has a diameter between about five millimeters and about thirty millimeters.

5. A colonoscopic device stabilizer as set forth in claim 1 wherein said body has a tapered tip for easing insertion into the anus.

6. A colonoscopic device stabilizer as set forth in claim 1 wherein the body has a thickness greater than a width of the colonoscopic device.

7. A colonoscopic device stabilizer as set forth in claim 1 wherein the opening in the body is circular.

8. A colonoscopic device stabilizer as set forth in claim 7 wherein the opening has a diameter between about five millimeters and about thirty millimeters.

9. A colonoscopic device stabilizer as set forth in claim 2 wherein the clamp comprises a cam lever.

10. A colonoscopic device stabilizer as set forth in claim 1 further comprising a strap extending from the body for attaching the stabilizer to the patient.

11. A colonoscopic device stabilizer as set forth in claim 10 wherein the strap at least partially encircles the patient when attaching the stabilizer to the patient.

12. A colonoscopic device stabilizer as set forth in claim 1 further comprising an adhesive strip extending from the body for attaching the stabilizer to the patient.

13. A method of inserting A colonoscopic device into an anus of a patient, said method comprising:
positioning a stabilizer into the anus of the patient;
inserting the colonoscopic device into the stabilizer; and
advancing the colonoscopic device through the stabilizer.

14. A method as set forth in claim 13 further comprising attaching the stabilizer to the patient.

15. A method as set forth in claim 14 wherein the attaching step is performed by strapping the stabilizer to the patient.

16. A method as set forth in claim 14 wherein the attaching step is performed by adhesively attaching the stabilizer to the patient.

17. A method as set forth in claim 13 further comprising clamping the colonoscopic device to the stabilizer once a tip of the device reaches a desired position within the patient.

18. A method of inserting an endoscopic device into an orifice in a patient, said method comprising:
positioning a stabilizer into the orifice of the patient;
inserting the endoscopic device into the stabilizer; and
advancing the endoscopic device through the stabilizer.

19. A method as set forth in claim 18 wherein the orifice is a natural orifice.

20. A method as set forth in claim 19 wherein the orifice is an anus of a patient.

21. A colonoscopic device stabilizer for stabilizing a colonoscopic device when inserted in an anus of a patient, said stabilizer comprising:
means for receiving the endoscopic device; and
means for selectively resisting movement of the endoscopic device relative to the receiving means.
